(19) United States
(12) Patent Application Publication
(10) Pub. No.: US 2010/0290982 A1
(43) Pub. Date: Nov. 18, 2010

(54) SOLID IN OIL/WATER EMULSION-DIFFUSION-EVAPORATION FORMULATION FOR PREPARING CURCUMIN-LOADED PLGA NANOPARTICLES

(75) Inventors: Amalendu Prakash Ranjan, Fort Worth, TX (US); Anindita Mukerjee, Fort Worth, TX (US); Jamboor K. Vishwanatha, Fort Worth, TX (US)

Correspondence Address: CHALKER FLORES, LLP, 2711 LBJ FRWY, Suite 1036, DALLAS, TX, 75234, US

(73) Assignee: University of North Texas Health Science Center at Fort Worth, Fort Worth, TX (US)

(21) Appl. No.: 12/766,068

(22) Filed: Apr. 23, 2010

Related U.S. Application Data
(63) Continuation-in-part of application No. 12/101,929, filed on Apr. 11, 2008.
(60) Provisional application No. 60/911,528, filed on Apr. 13, 2007.

(51) Int. Cl. A61K 51/02; A61K 31/713; A61K 39/395; A61K 31/121; A61P 35/00; A61P 31/10; A61P 31/12; A61P 33/10; A61P 31/00

(52) U.S. Cl. 424/1.11; 514/44 A; 424/130.1; 514/679; 977/773; 977/906; 977/882

(57) ABSTRACT

The present invention includes compositions and methods of making an activated polymeric nanoparticle for targeted drug delivery that includes a biocompatible polymer and an amphiphilic stabilizing agent non-covalently associated with a spacer compound that includes at least one electrophile that selectively reacts with any nucleophilic on a targeting agent and places the targeting agent on the exterior surface of a biodegradable nanoparticle, wherein an active agent is encapsulated in or about the nanoparticle.

### CROSS-REFERENCE TO RELATED APPLICATIONS

This application claims priority to U.S. Provisional Application Ser. No. 60/911,528, filed Apr. 13, 2007, and is a continuation in part of U.S. patent application Ser. No. 12/101,929, file Apr. 11, 2008, the entire contents of which are incorporated herein by reference.

### STATEMENT OF FEDERALLY FUNDED RESEARCH

This invention was made with U.S. Government support under Contract No. BCRP Concept BC075097 awarded by the Department of Defense. The government has certain rights in this invention.

### TECHNICAL FIELD OF THE INVENTION

The present invention relates in general to the field of active agent loaded particles, and more particularly, to compositions and methods for delivering active agents in PLGA loaded particles made by emulsion-diffusion evaporation (S-O/W) formulation with or without targeting agents.

### BACKGROUND OF THE INVENTION

Without limiting the scope of the invention, its background is described in connection with the delivery of active pharmaceutical agents.

One of the greatest problems associated with molecular therapeutics is delivery of the therapeutic agent to the site of action. For the case of anti-cancer agents, there is the necessity to keep the dose at minimal levels for the protection of the patient. The reduction in dose however; may not fully treat the disease. Thus, through the direction of a drug delivery device to a specific site of action via the conjugation of various antibodies, more advantageous therapeutic regimes can be developed.

### SUMMARY OF THE INVENTION

The present invention includes methods and compositions of making an optionally targetable, loadable-nanoparticle by Emulsion diffusion solvent evaporation comprising: (a) forming a first solution comprising a solvent, a polymer, and an active agent; (b) preparing a second solution comprising an amphiphilic stabilizing agent in water (including a spacer compound if targeted particle desired) (c) forming an emulsion by adding dropwise the 1st solution to the 2nd solution while sonicating to form an emulsion; (d) adding the emulsion formed in Step (c) into an excess of water with stirring for solvent diffusion and evaporation; (e) separate the nanoparticles from the emulsion formed in step (c) and (f) adding cryoprotectants to form active agent loaded nanoparticles. In one aspect, the first solution comprises PLGA and ethyl acetate. In another aspect, the second solution comprises 80% hydrolyzed PVA. In another aspect, the sonication time is between 30 second and 180 second, 45 seconds and 120 second, between 55 seconds and 90 seconds, and between 60 and 75 seconds. In another aspect, step (f) is followed by lyophilization. In another aspect, the method further comprises the addition of at least one of a targeting agent or a spacer in step (b). In another aspect, the method further comprises the addition of a spacer in step (b), wherein a targeting agent is attached to the spacer during or after any of step (b) through (f). In one aspect, the targeting agent is added after lyophilization.

In another aspect, the nanoparticles have a polydispersity of 0.130 to 0.160, 0.140 to 0.150. In another aspect, the method further comprises the step of drying the nanoparticles, wherein the nanoparticles form a dry homogenous particle. In another aspect, the emulsion is formed without any toxic solvents. In another aspect, the spacer is homofunctional, heterofunctional, multifunctional, monoreactive, bi-reactive or multireactive, water soluble, water-insoluble or partially water soluble. In another aspect, the spacer is defined further as comprising spacers have multiple lengths. In another aspect, the targeting agent is selected from an antibody, a small molecule, a peptide, a carbohydrate, a polysaccharide, a protein, a nucleic acid, an aptamer, a second nanoparticle, a cytokine, a chemokine, a lymphokine, a receptor, a lipid, a lectin, a ferrous metal, a magnetic particle, a linker, an isotope and combinations thereof. In another aspect, the active agent is selected from at least one of an anti-cancer drug, an antibiotic, an antiviral, an antifungal, an antihelminthic, a nutrient, a small molecule, a siRNA, an antioxidant, and an antibody. In another aspect, the active agent comprises a curcumin or curcuminoid. In another aspect, the targeting agent selectively targets the nanocarrier to diseased tissue/cells, thereby minimizing whole body dose. In another aspect, the nanoparticles are loaded with an active agent combines a conventional radioisotopes and a chemotherapeutic.

Another embodiment of the present invention is a pharmaceutical agent comprising: an activated polymeric nanoparticle for targeted drug delivery comprising a biocompatible polymer and an amphiphilic stabilizing agent non-covalently associated with a spacer compound comprising at least one electrophile that selectively reacts with a nucleophile on a targeting agent to bind the targeting agent on the exterior surface of a biodegradable nanoshell, wherein an active agent is loaded in the nanoshell and further comprising a pharmaceutically acceptable carrier, wherein the nanoshells are formed in a one-part emulsion without the use of toxic solvents.

Another embodiment of the present invention is a polymeric nanoparticle that is optionally targetable for drug delivery comprising: a biocompatible polymer and an amphiphilic stabilizing agent non-covalently associated with a spacer compound containing at least one electrophile that selectively reacts with a nucleophilic agent on a targeting agent to bind the targeting agent to an exterior surface of a biodegradable nanoshell, wherein an active agent is loaded with the nanoshell, wherein the nanoshells are formed in a one-part emulsion without the use of toxic solvents.

### BRIEF DESCRIPTION OF THE DRAWINGS

For a more complete understanding of the features and advantages of the present invention, reference is now made to the detailed description of the invention along with the accompanying figures and in which:

FIG. 1: Schematic diagram representing the S-O/W formulation for nanoparticle (targeted/untargeted) formation;

FIG. 2: Comparison between old and new formulation: Physical appearance of old and new formulation of nanoparticles;

FIG. 3: Comparison between old and new formulation: Redispersibility of old and new formulation of nanoparticles;

FIG. 4: a: Transmission electron micrograph of CUR-PLGA-NP; b: Scanning electron micrograph of CUR-PLGA-NP;

FIG. 5: Particle size distribution (batch A1) from New Example 1: Untargeted Curcumin loaded PLGA nanoparticles;

FIG. 6: Particle size distribution (batch A2) from New Example 1: Untargeted Curcumin loaded PLGA nanoparticles;

FIG. 7: Particle size distribution (batch A3) from New Example 1: Untargeted Curcumin loaded PLGA nanoparticles;

FIG. 8: Particle size distribution with linker (batch B1) from New Example 2: Targeted PLGA nanoparticles (linker: BS3);

FIG. 9: Particle size distribution with linker (batch B2) from New Example 2: Targeted PLGA nanoparticles (linker: BS3);

FIG. 10: Flow cytometry measurement of nanoparticles with antibody attachment. The majority of the nanoparticles were conjugated to the antibody as evidenced by a 92.8% antibody attachment using New Example 2: Targeted PLGA nanoparticles (linker: BS3);

FIG. 11: Particle size distribution with linker (batch C1) from New Example 3: Targeted PLGA nanoparticles (linker: s-EMCS);

FIG. 12: Particle size distribution with linker (batch C2) form New Example 3: Targeted PLGA nanoparticles (linker: s-EMCS);

FIG. 13: Flow cytometry measurement of nanoparticles with antibody attachment. The majority of the nanoparticles were conjugated to the antibody as evidenced by a 88.4% antibody attachment from New Example 3: Targeted PLGA nanoparticles (linker: s-EMCS);

FIG. 14: Particle size distribution with linker (batch D1) from New Example 4: Targeted Curcumin loaded PLGA nanoparticles (linker: BS3);

FIG. 15: Particle size distribution with linker (batch D2) from New Example 4: Targeted Curcumin loaded PLGA nanoparticles (linker: BS3);

FIG. 16: Particle size distribution with linker (batch D3) from New Example 4: Targeted Curcumin loaded PLGA nanoparticles (linker: BS3);

FIG. 17: Particle size distribution with antibody (batch D1) from New Example 4: Targeted Curcumin loaded PLGA nanoparticles (linker: BS3);

FIG. 18: Particle size distribution with antibody (batch D2) from New Example 4: Targeted Curcumin loaded PLGA nanoparticles (linker: BS3); and

FIG. 19: Flow cytometry measurement of nanoparticles with antibody attachment. The majority of the nanoparticles were conjugated to the antibody as evidenced by a 90.7% antibody attachment from New Example 4: Targeted Curcumin loaded PLGA nanoparticles (linker: BS3).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Detailed methodology for the formulation of activated curcumin loaded nanoparticles for secondary conjugation of biologically active agents (e.g. antibodies).

The present invention differs from existing technologies due to the fashion in which we target our particles. The most common method for the attachment of ligands to polymeric nanoparticles is through the grafting of poly ethylene glycol (PEG) to the PLGA polymeric strands thus making a PLGA-PEG copolymer. Linkage is performed to the PEG molecules using standard amine reactive chemistries. Our method generates an active particle for ligand attachment through the inclusion of a commercially available crosslinking agents (BS3, Pierce Biotechnology, Rockford, Ill.) present during the formation of the emulsion. The BS3 present within the emulsion solution is sequestered through hydrophobic/hydrophilic interactions between the PLGA emulsion and the PVA stabilizing agent also present in the emulsion solution.

Another advantage of the compositions and methods of the present invention is that any biologically active molecule with a nucleophilic group can be attached to the nanoshells and/or nanoparticles through reaction against an exposed NHS ester moiety (i.e., electrophile), leading to an unlimited range of targeted particles for therapeutic purposes.

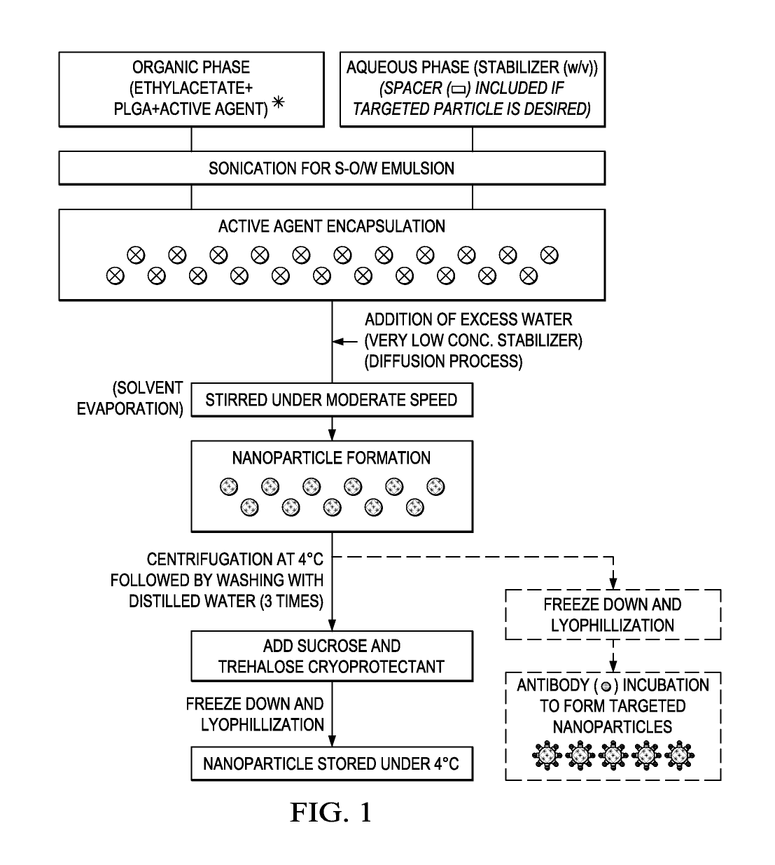

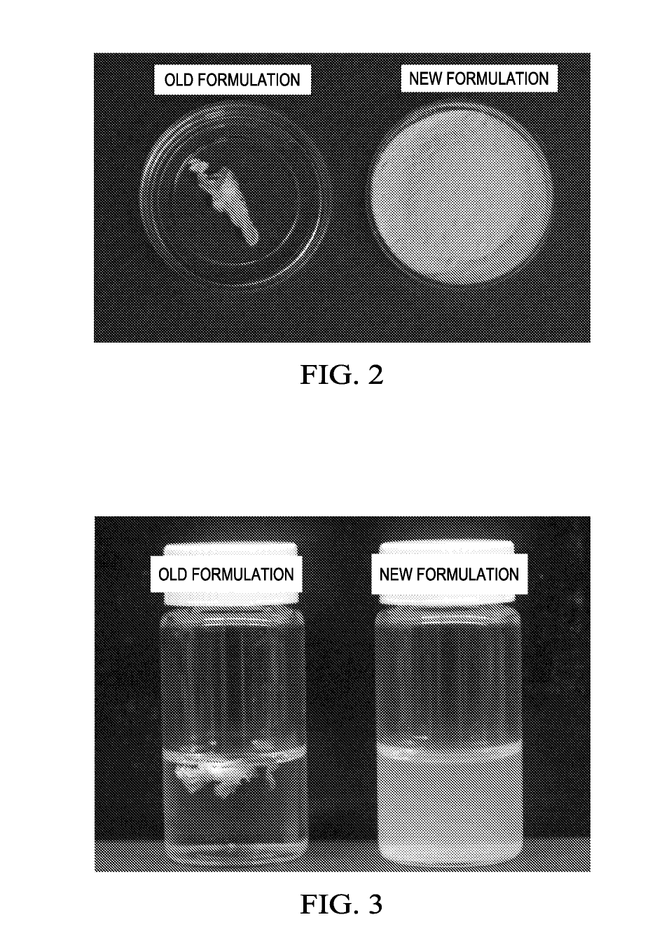

FIG. 1. Schematic diagram representing the S-O/W formulation for nanoparticle (targeted/untargeted) formation. FIG. 2: Physical appearance of old and new formulation of nanoparticles: Old formulation appears sheath like aggregated mass (very difficult to weigh and experiment); New Formulation appears as dry homogenous powder (can be accurately weighed and dosed).

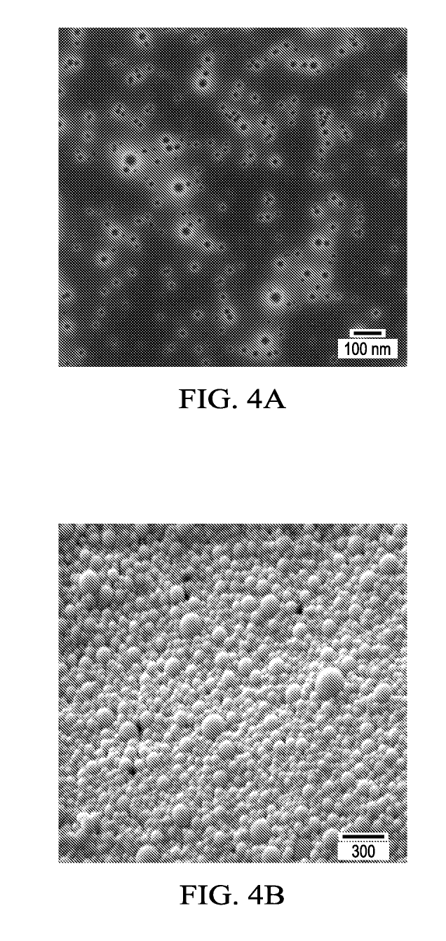

FIG. 3: Redispersibility of old and new formulation of nanoparticles: Redispersibility of lyophilized nanoparticles was improved many folds in New formulation. Old formulation resulted in a shealth like aggregate, which was difficult to redisperse in water. New Formulation resulted in nanoparticles could be redispersed in water by shaking FIG. 4a: Transmission electron micrograph of CUR-PLGA-NP; FIG. 4b: Scanning electron micrograph of CUR-PLGA-NP.

TABLE 1
Differences between old and new methods.

| | Our Previous Method | New Method |
|---|---|---|
| | (S/O/W Emulsion-Evaporation) | S-O/W (Emulsion-Diffusion-Evaporation) |
| Step 1 | Form 1st emulsion with solvent, | Form 1st solution with solvent, polymer, and |
| | polymer, and active agent; | active agent; |
| Step 2 | prepare 2nd solution with amphiphilic | prepare 2nd solution with amphiphilic |
| | stabilizing agent in water plus a non- | stabilizing agent in water [include a spacer |
| | solvent [include a spacer compound if | compound if targeted particle desired] |
| | targeted particle desired] | |
| Step 3 | form 2nd emulsion by mixing the 1st | form emulsion by adding dropwise the 1st |
| | emulsion and 2nd solution to form | solution to the 2nd solution while sonicating |
| | nanoparticles | to form emulsion |
| New | | emulsion formed in Step 3 is added to |
| Step | | excess of water (containing very low |
| | | concentration of stabilizer) with stirring for |
| | | solvent diffusion and evaporation |
| Step 4 | separate the nanoparticles from the | separate the nanoparticles from the |
| | emulsion formed in step 3 | emulsion formed in New Step |
| | | Cryoprotectants added before lyophilization |
| | | (in case of untargeted particles) |
| Step 5 | [if spacer compound used in Step 2, bind | [if spacer compound used in Step 2, bind a |
| | a targeting agent to the particle via the | targeting agent to the particle via the |
| | noncovalently associated spacer | noncovalently associated spacer compound] |
| | compound] | |

### Example 1

### Optimization of Curcumin Loaded PLGA Nanoparticle-Formulation Using Central Composite Design for Cancer Therapy

The objective of this study was to optimize and characterize curcumin-loaded poly (lactic acid-co-glycolic acid) nanoparticles (CUR-PLGA-NP) formulated using an emulsification-evaporation-solvent diffusion technique while determining the formulation variables like amount of PLGA, concentration of stabilizer and volume of organic phase and their influence the physiochemical properties of nanoparticles.

Curcumin is known to be a potent anti-cancer agent. However, the clinical potential of curcumin is limited by its poor bioavailability in physiochemical environment and short half life. Curcumin-loaded poly (lactic acid-co-glycolic acid) nanoparticles (CUR-PLGA-NP) were formulated using an emulsification-evaporation-solvent diffusion technique. The objective of this study was to optimize and characterize this formulation and determine the formulation variables like amount of PLGA, concentration of stabilizer and volume of organic phase and their influence the physiochemical properties of nanoparticles. The physiochemical properties of the developed formulations were evaluated were particle size, polydispersity, encapsulation efficiency and percentage drug loading.

A central composite design (CCD) was applied to optimize the CUR-PLGA-NP formulation. An analysis of variance was performed to determine response surfaces. Furthermore, the desirability function approach was applied to obtain the best-optimized condition among the multiple responses.

The optimal conditions for the preparation of CUR-PLGA-NP were determined for the amount of PLGA, percent concentration of PVA and volume of ethyl acetate. The encapsulation efficiency and percentage drug loading achieved at these optimal conditions were high, above 90% and 14% respectively. The mean particle size of the optimized batch was found to be less than 200 nm and polydispersity was 0.13. The optimized nanoparticles as examined by transmission electron microscopy (TEM) and scanning electron microscopy (SEM), were found to have a smooth and spherical surface. The in vitro studies proved that optimized CUR-PLGA-NP released in sustained manner over the period of 10 days. The cellular uptake study and bio-functional assay showed the integrity of the drug incorporated in the nanoparticle. Stability analysis for a period of 90 days was performed and particle size analysis, encapsulation efficiency and drug loading were studied. The results revealed long term physiochemical stability of the CUR-PLGA-NP formulation.

These results demonstrate that the CCD design facilitated the optimization of CUR-PLGA-NP carrier systems for understanding the effect of formulation composition and sustained delivery of the drug for its use as an adjunct with cancer therapy to improve its efficacy.

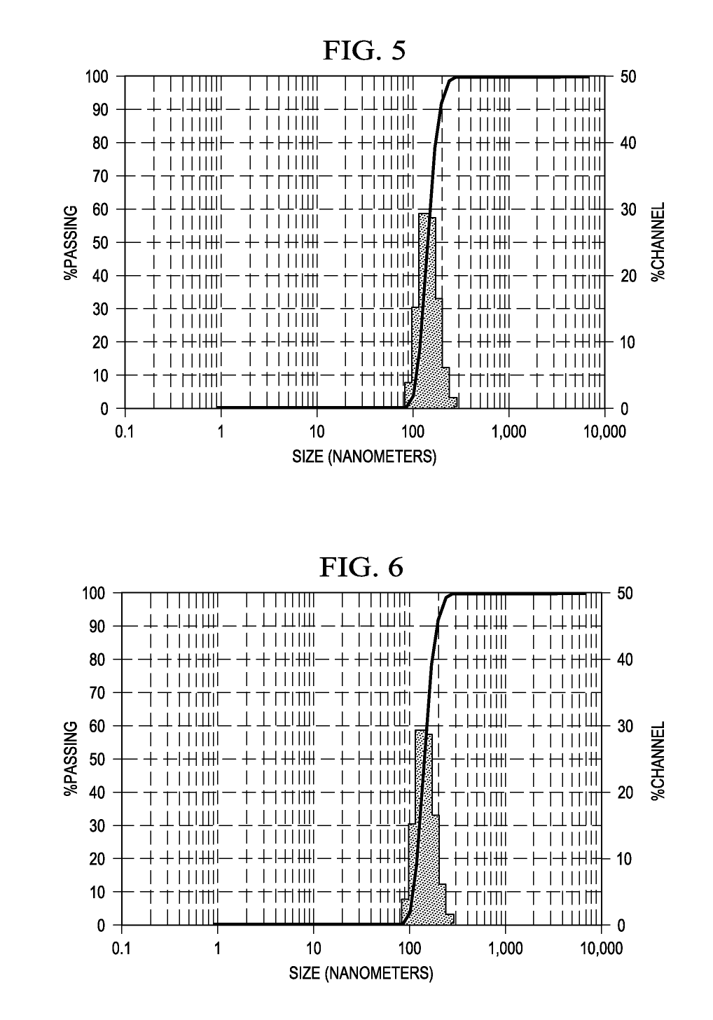

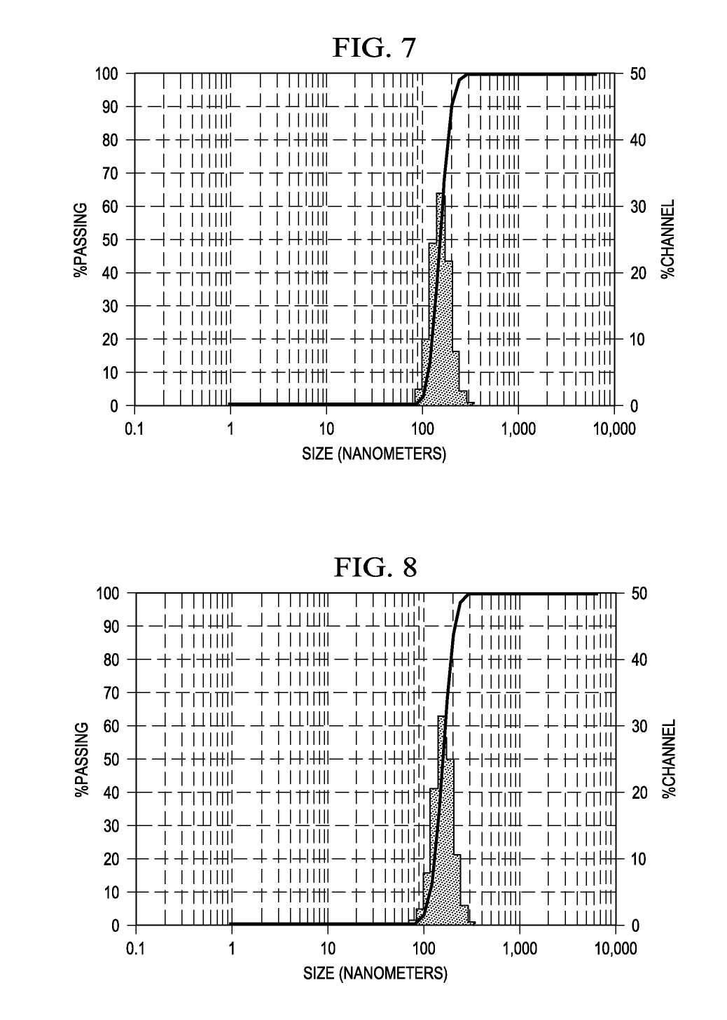

The results obtained with New Example 1 as depicted in the following figures. FIG. 5: Particle size distribution (mean particle size=146.4 nm) [batch A1]. FIG. 6: Particle size distribution (mean particle size=148.7 nm) [batch A2]. FIG. 7: Particle size distribution (mean particle size=155.6 nm) [batch A3].

TABLE 2

| | Our Previous Method | New Example 1 |
|---|---|---|
| | (S/O/W Emulsion-Evaporation) | Untargeted Particle |
| Step 1 | Form 1st emulsion with solvent, | 85 mg of PLGA is dissolved in 4.5 ml of ethyl |
| | polymer, and active agent; | acetate. Curcumin is added to this solution and |
| | | dispersed. No sonication |
| Step 2 | prepare 2nd solution with | 1% (w/v) PVA (80% hydrolysed) is prepared (2^{nd} |
| | amphiphilic stabilizing agent in | solution). No use of non-solvent. [include a spacer |
| | water plus a non-solvent [include | compound if targeted particle desired] |
| | a spacer compound if targeted | |
| | particle desired] | |
| Step 3 | form 2nd emulsion by mixing the | PLGA/curcumin/ethyl acetate solution is added |
| | 1st emulsion and 2nd solution to | dropwise to 10 ml of 1% PVA. Sonication time: 1 |
| | form nanoparticles | minute^{##} [^{##} Less sonication time is |
| | | beneficial for RNA/DNA encapsulation] |
| New | | Emulsion formed in step 3 is added to excess of |
| Step | | water with 0.1% PVA (40 ml) and stirred on a |
| | | magnetic stirrer for 4-6 hours to bring about |
| | | diffusion and complete solvent evaporation |
| Step 4 | separate the nanoparticles from | Nanoparticles thus formed are separated by |
| | the emulsion formed in step 3 | centrifugation at 12000 rpm for 45 minutes. |
| | | Nanospheres are washed 3X with DD nanofilter |
| | | (0.2 μm)water. Cryoprotectants: trehalose and |
| | | sucrose(5:1)are added to the nanoparticles before |
| | | freeze drying and lyophilization |
| Step 5 | [if spacer compound used in Step | |
| | 2, bind a targeting agent to the | |
| | particle via the noncovalently | |
| | associated spacer compound] | |
| | Payload | curcumin |
| | Spacer/Linker | none |
| | Targeting agent | none |
| | RESULTS | New Example 1 |
| | Percent Yield | 90.3% ± 1.2% |
| | Encapsulation Efficiency | 91.4% ± 3.4% |
| | Particle Size | 150.3 ± 5 nm |
| | Polydispersity | smaller particle size range, narrow size |
| | | distribution and low polydispersity of 0.131 ± .005 |
| | Appearance/Surface Morphology | smooth and spherical nanoparticles as observed in |
| | | TEM and SEM scans |
| | Redispersibility in water | Redispersibility of lyophilized nanoparticles was |
| | | improved many folds. Nanoparticles could be |
| | | redispersed in water by shaking without use of |
| | | any ultrasonic water bath |
| | Toxicity | No cell death observed with new formulation; no |
| | | etch marks observed on tissue culture plates |
| | In vitro Release Profile | intial burst phase - 10-13% in 30 min; sustained |
| | | release - 75%/10 days |
| | Celluar Uptake | robust endocytosis by prostate and breast cancer |
| | | cell lines: DU145, MDA MB231, MCF-7 & MCF-10A |
| | Stability | Stability studies have been carried out for 6 |
| | | months and the studies show that nanoparticles |
| | | formulated by this new method is stable for the |
| | | entire study duration. No significant change |
| | | observed in particle size, drug loading and |
| | | morphology of particles when stored at 4° C. |
| | Scalability | The New formulation has been successfully |
| | | optimized and scaled to produce 5 gm (or more) |
| | | batches |
| | Ease of Handling | Dry homogenous powder; very easy to weigh and |
| | | use |
| | Dose Calculation | The new formulation can be measured accurately |
| | | and dosed |

### Example 2

### Evaluation of Annexin A2 Antibody-Conjugated Curcumin-Loaded Nanospheres as Targeted Drug Carrier Systems for Breast Cancer Therapy

Among the potent anti-cancer agents, curcumin has been found to be very effective against various cancer cells. In our present study, we formulated annexin A2 antibody conjugated poly lactic-co-glycolic acid (PLGA) nanospheres for targeted delivery of curcumin to breast cancer cells.

Targeting anticancer drugs to their specific molecular targets is still a major challenge in cancer therapy. Among the potent anti-cancer agents, curcumin has been found to be very efficacious against various cancer cells. In our present study, we formulated annexin A2 antibody conjugated poly lactic-co-glycolic acid (PLGA) nanospheres for targeted delivery of curcumin to breast cancer cells.

The nanospheres were formulated using solid/oil/water emulsion solvent evaporation method and then characterized for percent yield, encapsulation efficiency, surface morphology, particle size, drug distribution within nanospheres and drug polymer interaction. Functionalized nanospheres for antibody conjugation was prepared using a cross-linking ligand, bis(sulfosuccinimidyl) suberate (BS3), which conjugated efficiently to the primary amino groups of the antibody.

These studies showed the successful formation of smooth and spherical curcumin loaded PLGA nanospheres with a high percent yield of about 90.01±0.13% and an encapsulation efficiency of 90.28±0.14%. The mean particle size of the nanospheres was found to be 145 nm. The percent antibody attachment to PLGA nanospheres was found to be 92.8%. The in vitro drug release profile showed 60% drug release from the nanospheres in 24 hours. Results showed robust intra-cellular uptake of the nanospheres in the cells. Cell viability studies revealed that these curcumin loaded nanospheres resulted in less cell viability for the cancer cells as compared to normal cell line.

These studies show successful formulation of annexin A2 conjugated curcumin loaded PLGA nanospheres. Intracellular uptake and cell viability assays demonstrated efficient targeting, uptake and action of curcumin nanospheres in breast cancer cell lines. The effectiveness of this nanoparticlute carrier system for targeted delivery of anticancer drugs has a potential to improve the efficacy of therapy in patients with breast cancer.

TABLE 3
New Example with Targeted Nanoparticles

| | Our Previous Method | New Example 2 |
|---|---|---|
| | (S/O/W Emulsion-Evaporation) | Targeted Particle |
| Step 1 | Form 1st emulsion with solvent, | 85 mg of PLGA is dissolved in 4.5 ml of ethyl |
| | polymer, and active agent; | acetate. No sonication |
| Step 2 | prepare 2nd solution with amphiphilic | 1% (w/v) PVA (80% hydrolysed) is prepared |
| | stabilizing agent in water plus a non- | (2^{nd} solution). No use of non-solvent. A |
| | solvent [include a spacer compound if | spacer/linker compound is added to prepare |
| | targeted particle desired] | targeted particles |
| Step 3 | form 2nd emulsion by mixing the 1st | PLGA/curcumin/ethyl acetate solution is |
| | emulsion and 2nd solution to form | added dropwise to 10 ml of 1% PVA with |
| | nanoparticles | spacer/linker. Sonication time: 1 minute |
| New | | Emulsion formed in step 3 is added to excess |
| Step | | of water with 0.1% PVA (20 ml) and stirred on |
| | | a magnetic stirrer for 4-6 hours to bring about |
| | | diffusion and complete solvent evaporation |
| Step 4 | separate the nanoparticles from the | Nanoparticles thus formed were separated by |
| | emulsion formed in step 3 | centrifugation at 10000 rpm for 30 minutes. |
| | | Nanospheres were washed 3X with DD |
| | | nanofilter (0.2 mm)water. |
| Step 5 | [if spacer compound used in Step 2, | Nanoparticles bound with spacer/linker were |
| | bind a targeting agent to the particle | incubated with targeting agent (antibody) for |
| | via the noncovalently associated | overnight. |
| | spacer compound] | |
| | Payload | none |
| | Spacer/Linker | BS3 (11.4 A; homo functionality) |
| | Targeting agent | Annexin A2 |
| | RESULTS | New Example 2 |
| | Percent Yield | 86.4% ± 2.7% |
| | Encapsulation Efficiency | |
| | Particle Size | 162.7 ± 3.2 nm (NP-linker) |
| | Polydispersity | smaller particle size range, narrow size |
| | | distribution |
| | Appearance/Surface Morphology | smooth and spherical nanoparticles as |
| | | observed in TEM scans |
| | | 92.8%. |
| | Redispersibility in water | Redispersibility of lyophilized nanoparticles |
| | | was improved many folds. Nanoparticles |
| | | could be redispersed in water by shaking |
| | | without use of any ultrasonic water bath |
| | Celluar Uptake | improved targeting seen in MDA MB231 brest |
| | | cancer cells (qualitative only) |
| | Ease of Handling | Dry homogenous powder; very easy to weigh |
| | | and use |
| | Dose Calculation | This new formulation can be measured |
| | | accurately and dosed |

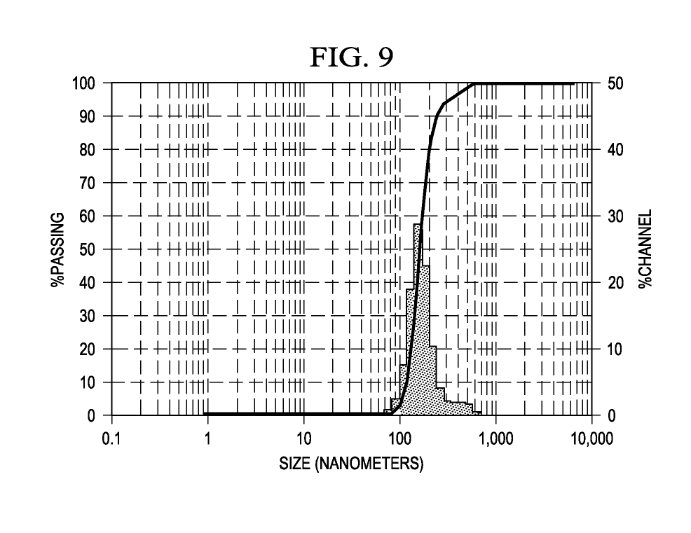

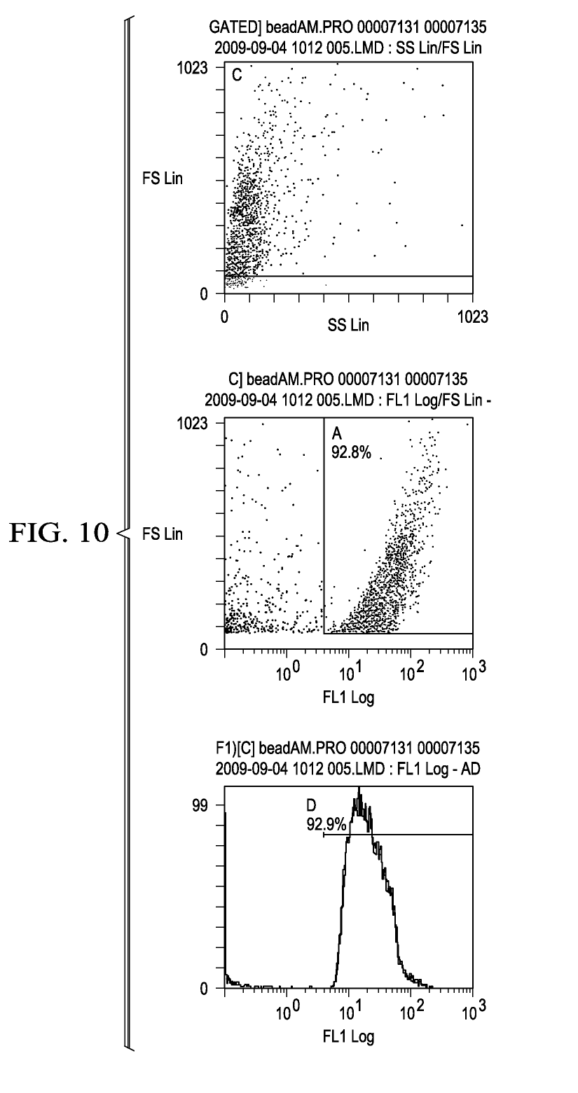

The results obtained with New Example 2 are as follows. FIG. 8: Particle size distribution (mean particle size=161.2 nm) [batch B1] with linker. FIG. 9: Particle size distribution (mean particle size=164.3 nm) [batch B2] with linker. FIG. 10: Flow cytometry measurement of nanoparticles with antibody attachment. The majority of the nanoparticles were conjugated to the antibody as evidenced by a 92.8% antibody attachment.

TABLE 4
Targeted Nanoparticle

| | Our Previous Method | New Example 3 |
|---|---|---|
| | (S/O/W Emulsion-Evaporation) | Targeted Particle |
| Step 1 | Form 1st emulsion with solvent, | 85 mg of PLGA is dissolved in 4.5 ml of ethyl |
| | polymer, and active agent; | acetate.. No sonication |
| Step 2 | prepare 2nd solution with amphiphilic | 1% (w/v) PVA (80% hydrolysed) is prepared |
| | stabilizing agent in water plus a non- | (2^{nd} solution). No use of non-solvent. A |
| | solvent [include a spacer compound if | spacer/linker compound is added to prepare |
| | targeted particle desired] | targeted particles |
| Step 3 | form 2nd emulsion by mixing the 1st | PLGA/curcumin/ethyl acetate solution is |
| | emulsion and 2nd solution to form | added dropwise to 10 ml of 1% PVA with |
| | nanoparticles | spacer/linker. Sonication time: 1 minute |
| New | | Emulsion formed in step 3 is added to excess |
| Step | | of water with 0.1% PVA (20 ml) and stirred on |
| | | a magnetic stirrer for 4-6 hours to bring about |
| | | diffusion and complete solvent evaporation |
| Step 4 | separate the nanoparticles from the | Nanoparticles thus formed were separated by |
| | emulsion formed in step 3 | centrifugation at 10000 rpm for 30 minutes. |
| | | Nanospheres were washed 3X with DD |
| | | nanofilter (0.2 mm)water. |
| Step 5 | [if spacer compound used in Step 2, | Nanoparticles bound with spacer/linker were |
| | bind a targeting agent to the particle | incubated with targeting agent (antibody) for |
| | via the noncovalently associated | overnight. |
| | spacer compound] | |
| | Payload | none |
| | Spacer/Linker | s-EMCS (9.4 A; hetero functionality) |
| | Targeting agent | Annexin A2 |
| | RESULTS | New Example 3 |
| | Percent Yield | 85.1% ± 3.1% |
| | Encapsulation Efficiency | |
| | Particle Size | 160.1 ± 2.1 nm (NP-linker) |
| | Polydispersity | smaller particle size range, narrow size |
| | | distribution |
| | Appearance/Surface Morphology | smooth and spherical nanoparticles as |
| | | observed in TEM scans |
| | | 88.4%. |
| | Redispersibility in water | Redispersibility of lyophilized nanoparticles |
| | | was improved many folds. Nanoparticles |
| | | could be redispersed in water by shaking |
| | | without use of any ultrasonic water bath |
| | Ease of Handling | Dry homogenous powder; very easy to weigh |
| | | and use |
| | Dose Calculation | This new formulation can be measured |
| | | accurately and dosed |

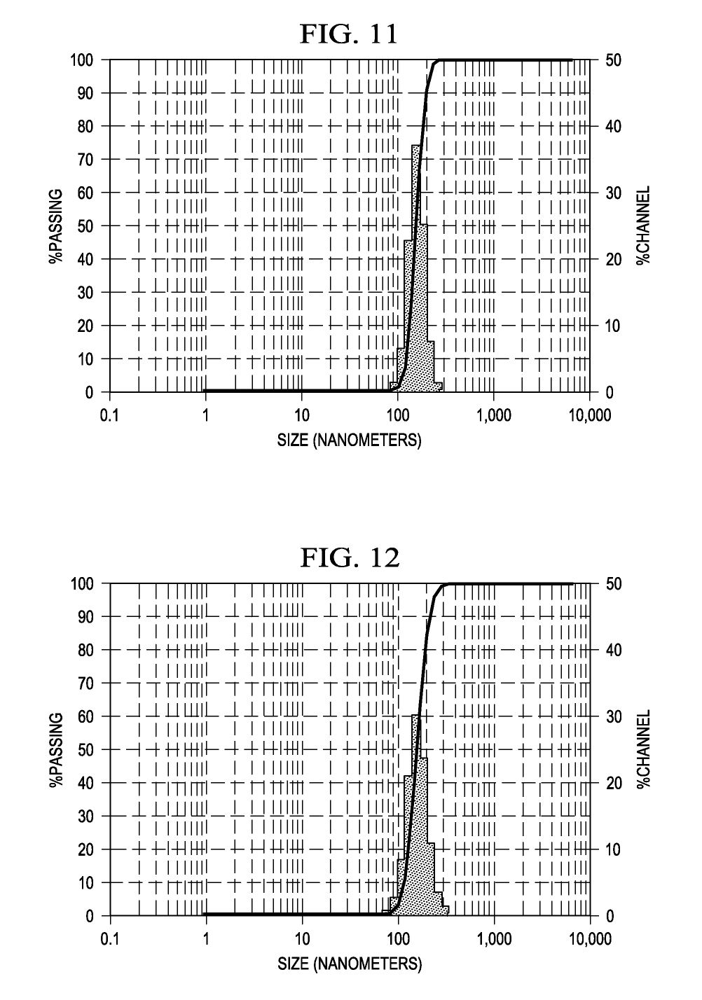

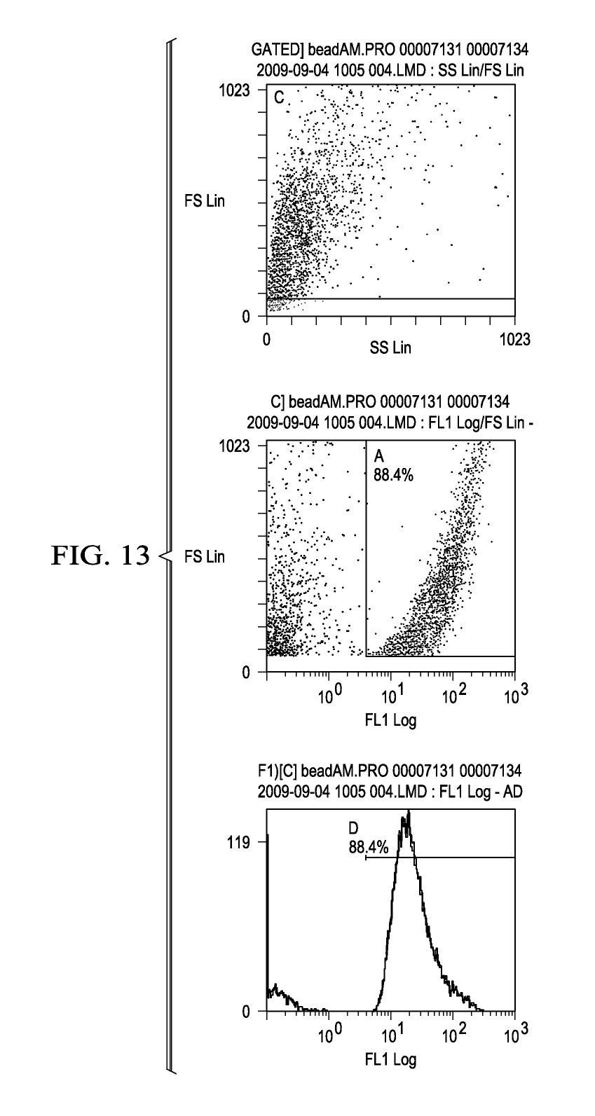

The results obtained with New Example 3 are as follows. FIG. 11: Particle size distribution (mean particle size=159.1 nm) [batch C1] with linker. FIG. 12: Particle size distribution (mean particle size=160.9 nm) [batch C2] with linker. FIG. 13: Flow cytometry measurement of nanoparticles with antibody attachment. The majority of the nanoparticles were conjugated to the antibody as evidenced by a 88.4% antibody attachment.

TABLE 5

| | Our Previous Method | New Example 4 |
|---|---|---|
| | (S/O/W Emulsion-Evaporation) | Targeted Particle |
| Step 1 | Form 1st emulsion with solvent, | 85 mg of PLGA is dissolved in 4.5 ml of ethyl |
| | polymer, and active agent; | acetate. Curcumin is added to this solution |
| | | and dispersed. No sonication |
| Step 2 | prepare 2nd solution with amphiphilic | 1% (w/v) PVA (80% hydrolysed) is prepared |
| | stabilizing agent in water plus a non- | (2^{nd} solution). No use of non-solvent. A |
| | solvent [include a spacer compound if | spacer/linker compound was added to |
| | targeted particle desired] | prepare targeted particles |
| Step 3 | form 2nd emulsion by mixing the 1st | PLGA/curcumin/ethyl acetate solution is |
| | emulsion and 2nd solution to form | added dropwise to 10 ml of 1% PVA with |
| | nanoparticles | spacer/linker. Sonication time: 1 minute |
| New | | Emulsion formed in step 3 is added to excess |
| Step | | of water with 0.1% PVA (20 ml) and stirred on |
| | | a magnetic stirrer for 4-6 hours to bring about |
| | | diffusion and complete solvent evaporation |
| Step 4 | separate the nanoparticles from the | Nanoparticles thus formed were separated by |
| | emulsion formed in step 3 | centrifugation at 10000 rpm for 30 minutes. |
| | | Nanospheres were washed 3X with DD |
| | | nanofilter (0.2 mm)water. |
| Step 5 | [if spacer compound used in Step 2, | Nanoparticles bound with spacer/linker were |
| | bind a targeting agent to the particle | incubated with targeting agent (antibody) for |
| | via the noncovalently associated | overnight. |
| | spacer compound] | |
| | Payload | curcumin |
| | Spacer/Linker | BS3 (11.4 A; homo functionality) |
| | Targeting agent | Annexin A2 |
| | RESULTS | New Example 4 |
| | Percent Yield | 87.6% ± 1.8% |
| | Encapsulation Efficiency | 89.1% ± 1.4% |
| | Particle Size | 180.8 ± 3.4 nm (NP-linker) 205 nm (with |
| | | antibody) |
| | Polydispersity | smaller particle size range, narrow size |
| | | distribution |
| | Appearance/Surface Morphology | smooth and spherical nanoparticles as |
| | | observed in TEM scans |
| | | 90.7%. |
| | Redispersibility in water | Redispersibility of lyophilized nanoparticles |
| | | was improved many folds. Nanoparticles |
| | | could be redispersed in water by shaking |
| | | without use of any ultrasonic water bath |
| | Toxicity | No cell death observed with new formulation; |
| | | no etch marks observed on tissue culture |
| | | plates |
| | Ease of Handling | Dry homogenous powder; very easy to weigh |
| | | and use |
| | Dose Calculation | This new formulation can be measured |
| | | accurately and dosed |

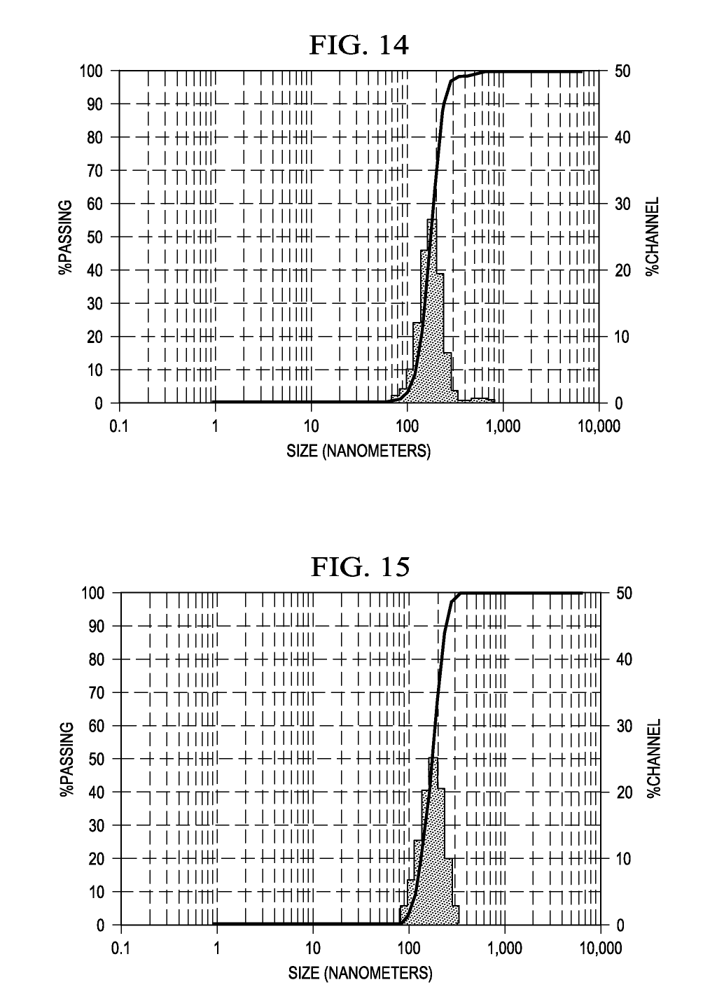

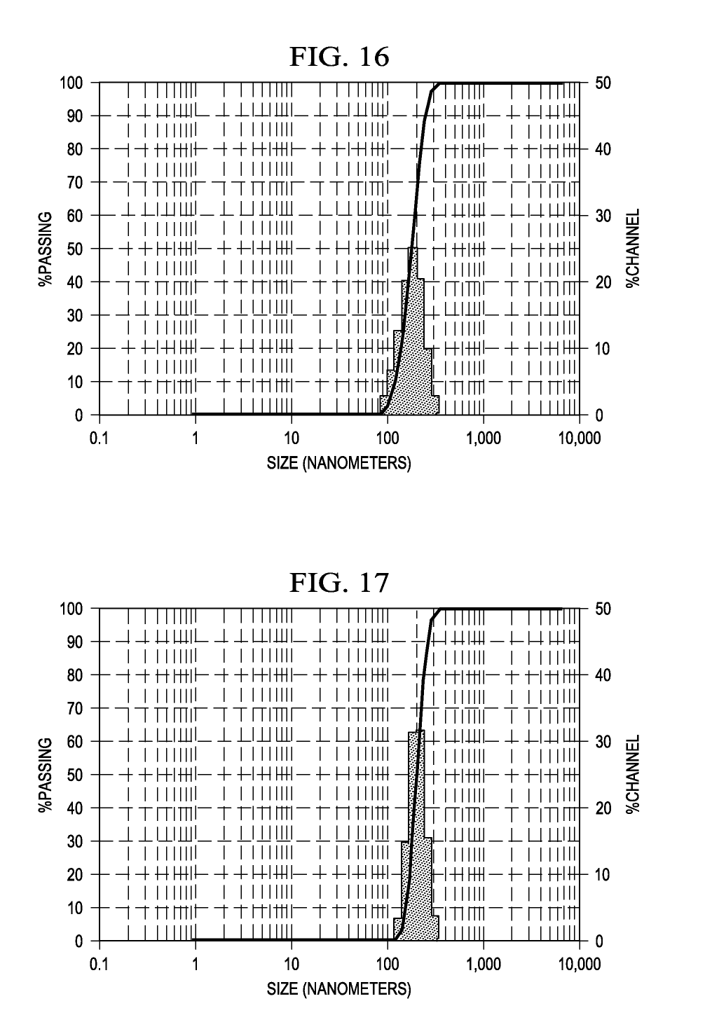

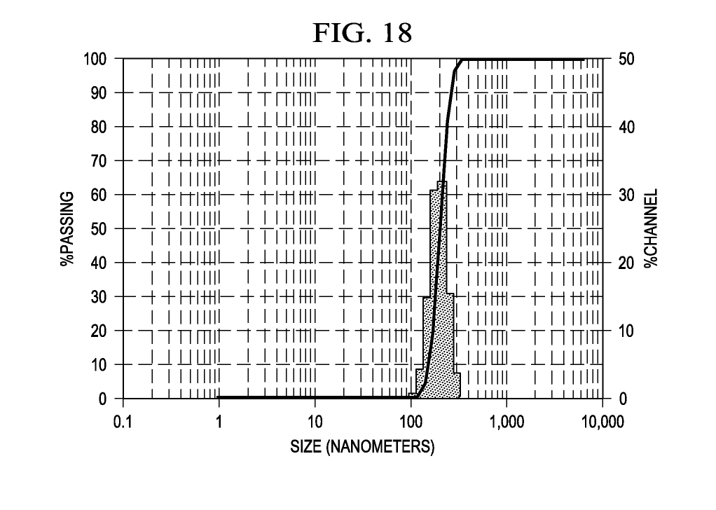

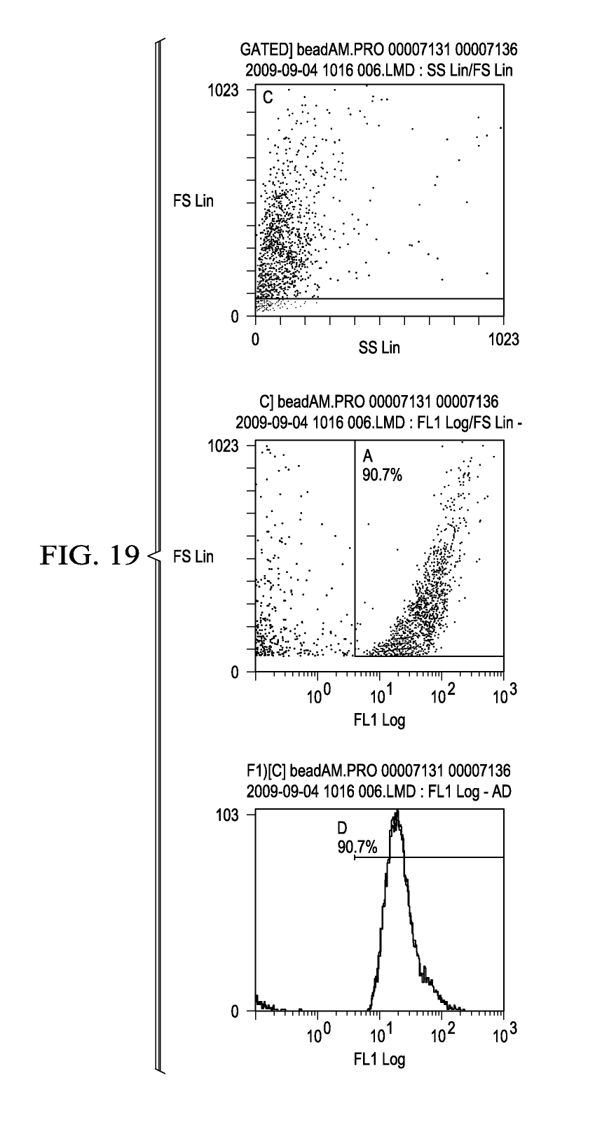

The results obtained with New Example 4 are as follows. FIG. 14: Particle size distribution (mean particle size=180.2 nm) [batch D1] with linker. FIG. 15: Particle size distribution (mean particle size=180.4 nm) [batch D2] with linker. FIG. 16: Particle size distribution (mean particle size=181.8 nm) [batch D3] with linker. FIG. 17: Particle size distribution (mean particle size 204.9 nm) [batch D1] with antibody. FIG. 18: Particle size distribution (mean particle size=205.0 nm) [batch D2] with antibody. FIG. 19: Flow cytometry measurement of nanoparticles with antibody attachment. The majority of the nanoparticles were conjugated to the antibody as evidenced by a 90.7% antibody attachment.

### Example 3

### Formulation and Characterization of Antibody Coated Poly(Lactic-Co-Glycolic Acid) to Target Metastatic Cancer

The treatment of cancer is limited by the side effects of the anti-cancer drugs. To overcome this problem it is important to deliver the drug at the site of cancer in the body in right amount. A novel way to approach this problem is through targeted drug delivery system, which will preferentially deliver the drug to the site of cancer. The objective of this project was to use antibodies that recognize the cancer cells and to direct the drug containing tiny spherical particles (nanoparticles) to the cancer cells.

Chemotherapy is the only available option for the treatment of advanced cancers. However, increasing evidences of drug resistance and non-specific toxicity of these agents limits their therapeutic outcomes. The objective of this project is to develop nanoparticle mediated targeted therapies to overcome these problems.

We used solid/oil/water (s/o/w) method to formulate curcumin encapsulating poly(lactic-co-glycolic acid) (PLGA) nanoparticles (NPs) functionalized with Bis(Sulfosuccinimidyl) suberate (BS3) to attach annexin A2 antibody. We further used Box Behnken Design (BBD) to optimize the formulation for different parameters. We characterized these NPs for particle size, stability, polydispersity index, zeta potential and surface morphology. We used flow cytometry to evaluate efficiency of antibody attachment. We studied the in-vitro release kinetics of nanoparticles as well as effect of sustained release of curcumin on nuclear factor kappa-light-chain-enhancer of activated B cells (NF-κB) localization in MDA-MB-231 cells.

The factorial design used for optimization provided us the optimal formulation parameters. The average size of these NPs was 184.1±17.9 nm and 193.8±21.34 nm before and after attachment of antibody respectively. The particles were spherical in shape and we found that antibodies are attached on the surface of nanoparticles. The antibody-coated NPs have nearly neutral surface charge and are readily taken up by the cell. The cytometric analysis showed that approximately 87.5% of NPs were coated with antibody. We found that approximately 76% of drug is released form matrix in 9 days it follows the Higuchi square root model of release kinetics form matrix formulation where diffusion is the major process for drug release. We also found that the nanoparticles caused sustained inhibition of p65 (NF-κB) translocation to nucleus over the time as compared to free drug.

From the results obtained in this study, it was concluded that the antibodies can be efficiently attached on the surface of nanoparticles using the BS-3 chemical crosslinker. The curcumin encapsulating nanoparticles also has sustained released properties, which can inhibit NF-κB for longer duration than just the free drug. Therefore, the antibody coated nanoparticles can be used a novel therapeutic approach in treatment of cancer.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method of making an optionally targetable, loadable-nanoparticle by Emulsion diffusion solvent evaporation comprising:
(a) forming a first solution comprising a solvent, a polymer, and an active agent;
(b) preparing a second solution comprising an amphiphilic stabilizing agent in water;
(c) forming an emulsion by adding dropwise the 1st solution to the 2nd solution while sonicating to form an emulsion;
(d) adding the emulsion formed in Step (c) into an excess of water with stirring for solvent diffusion and evaporation;
(e) separate the nanoparticles from the emulsion formed in step (c) and
(f) adding cryoprotectants to form active agent loaded nanoparticles.

2. The method of claim 1, wherein the first solution comprises PLGA and ethyl acetate.

3. The method of claim 1, wherein the second solution comprises 80% hydrolyzed PVA.

4. The method of claim 1, wherein the sonication time is between 30 second and 180 second, 45 seconds and 120 second, between 55 seconds and 90 seconds, and between 60 and 75 seconds.

5. The method of claim 1, wherein step (f) is followed by lyophilization.

6. The method of claim 1, further comprising the addition of at least one of a targeting agent or a spacer in step (b).

7. The method of claim 1, further comprising the addition of a spacer in step (b), wherein a targeting agent is attached to the spacer during or after any of step (b) through (f) or after lyophilization.

8. The method of claim 1, wherein the nanoparticles have a polydispersity of 0.130 to 0.160, 0.140 to 0.150.

9. The method of claim 1, further comprising the step of drying the nanoparticles, wherein the nanoparticles form a dry homogenous powder.

10. The method of claim 1, wherein the emulsion is formed without any toxic solvents.

11. The method of claim 1, wherein the spacer is homofunctional, heterofunctional, multifunctional, monoreactive, bi-reactive or multireactive, water soluble, water-insoluble or partially water-soluble.

12. The method of claim 1, wherein the spacer is defined further as comprising spacers have multiple lengths.

13. The method of claim 1, wherein the targeting agent is selected from an antibody, a small molecule, a peptide, a carbohydrate, a polysaccharide, a protein, a nucleic acid, an aptamer, a second nanoparticle, a cytokine, a chemokine, a lymphokine, a receptor, a lipid, a lectin, a ferrous metal, a magnetic particle, a linker, an isotope and combinations thereof.

14. The method of claim 1, wherein the active agent is selected from at least one of an anti-cancer drug, an antibiotic, an antiviral, an antifungal, an antihelminthic, a nutrient, a small molecule, a siRNA, an antioxidant, and an antibody.

15. The method of claim 1, wherein the active agent comprises a curcumin or curcuminoid.

16. The method of claim 1, wherein the targeting agent selectively targets the nanocarrier to diseased tissue/cells, thereby minimizing whole body dose.

17. The method of claim 1, wherein the nanoparticles are loaded with an active agent combines a conventional radioisotopes and a chemotherapeutic.

18. A nanoparticle made by the method of claim 1.

19. A pharmaceutical agent comprising:
an activated polymeric nanoparticle for targeted drug delivery comprising a biocompatible polymer and an amphiphilic stabilizing agent non-covalently associated with a spacer compound comprising at least one electrophile that selectively reacts with a nucleophile on a targeting agent to bind the targeting agent on the exterior surface of a biodegradable nanoshell, wherein an active agent is loaded in the nanoshell and further comprising a pharmaceutically acceptable carrier, wherein the nanoshells are formed in a single emulsion without the use of toxic solvents.

20. A polymeric nanoparticle that is optionally targetable for drug delivery comprising:
a biocompatible polymer and an amphiphilic stabilizing agent non-covalently associated with a spacer compound containing at least one electrophile that selectively reacts with a nucleophilic agent on a targeting agent to bind the targeting agent to an exterior surface of a biodegradable nanoshell, wherein an active agent is loaded with the nanoshell, wherein the nanoshells are formed in a single emulsion without the use of toxic solvents.

21. The nanoparticle of claim 20, wherein the nanoshell comprises one or more polyesters and one or more amphiphilic stabilizing agents.

22. The nanoparticle of claim 21, wherein the polyester is poly-lactic acid, poly glycolic acid, poly-lactic-co-glycolic acid, and combinations thereof.

23. The nanoparticle of claim 21, wherein the amphiphilic stabilizing agent is a polyol.

24. The nanoparticle of claim 21, wherein the polyol at least one of polyvinyl alcohol, polyethylene glycol, polypropylene glycol, polypropylenediol, polytetrahydrofuran or poly(ethylene oxide)-polypropylene oxide)-poly(ethylene oxide) (PEO-PPO-PEO) triblock copolymers.

25. The nanoparticle of claim 20, wherein the nanoshell encapsulates an active agent.

26. The nanoparticle of claim 20, wherein nanoshell composition is used to control the ultimate size and drug delivery rate.

27. The nanoparticle of claim 20, wherein the targeting agent selectively targets the nanocarrier to diseased tissue/cells, thereby minimizing whole body dose.

28. The nanoparticle of claim 20, wherein the nanoshell loaded with an active agent combines a conventional radioisotopes and a chemotherapeutic.

29. The nanoparticle of claim 20, wherein the nanoshell is adapted for controlled release of the active agents by pre-determining the polymeric ratios of lactic to glycolic acid.

30. The nanoparticle of claim 20, wherein the spacer is homofunctional, heterofunctional, multifunctional, monoreactive, bi-reactive or multireactive, water soluble, water-insoluble or partially water-soluble.

31. The nanoparticle of claim 20, wherein the spacer is defined further as comprising spacers have multiple lengths.

32. The nanoparticle of claim 20, wherein the targeting agent is selected from the group consisting of an antibody, a small molecule, a peptide, a carbohydrate, an siRNA, a protein, a nucleic acid, an aptamer, a second nanoparticle, a cytokine, a chemokine, a lymphokine, a receptor, a lipid, a lectin, a ferrous metal, a magnetic particle, a linker, an isotope and combinations thereof.

33. The nanoparticle of claim 20, wherein the active agent is selected from at least one of an anti-cancer drug, an antibiotic, an antiviral, an antifungal, an antihelminthic, a nutrient, a small molecule, a siRNA, an antioxidant, and an antibody.

34. The nanoparticle of claim 20, wherein the active agent comprises a curcumin or curcuminoid.
